(19) United States
(12) Patent Application Publication
(10) Pub. No.: US 2026/0234684 A1
(43) Pub. Date: Aug. 13, 2026

(54) CELL-FREE REPLICATION OF A NUCLEIC ACID CONSTRUCT

(71) Applicant: Novel Biotechnology USA, Inc., Vancouver, WA (US)

(72) Inventor: Matthew WEINSTOCK, Camas, WA (US)

(21) Appl. No.: 19/434,624

(22) Filed: Dec. 29, 2025

Related U.S. Application Data
(63) Continuation of application No. PCT/US24/36207, filed on Jun. 28, 2024.
(60) Provisional application No. 63/511,373, filed on Jun. 30, 2023.

(51) Int. Cl. C12P 19/34

(52) U.S. Cl. CPC C12P 19/34

(57) ABSTRACT

The present disclosure provides methods for cell free replication of nucleic acid constructs from template circular nucleic acid constructs that comprises rolling circle amplification of the template circular nucleic acid constructs to produce a replication product. In some instances, the present disclosure provides methods of producing a replicated circular DNA construct from the replication product using a recombinase. In some instances, the present disclosure provides methods of producing a replicated linear RNA construct from the replication product by digesting the replication product and performing in vitro transcription of the digested replication product. The disclosure also provides kits for cell-free replication of said nucleic acid constructs.

### CROSS REFERENCE

This application is a continuation of PCT/US2024/36207, filed Jun. 28, 2024, which claims the benefit of U.S. Provisional Application No. 63/511,373, filed on Jun. 30, 2023, each of which is incorporated by reference in its entirety.

### SUMMARY

Disclosed herein are in vitro methods of replicating a nucleic acid construct (such as a DNA construct or an RNA construct) from a template circular nucleic acid construct.

One aspect the invention features an in vitro method of producing a replicated circular DNA construct or a replicated linear RNA construct from a template circular nucleic acid construct in a cell-free system, where the template nucleic acid construct comprises a target sequence, one or more enzyme recognition sites flanking one or more ends of the target sequence, and a backbone sequence. The method comprising contacting the template circular nucleic acid construct with a DNA polymerase configured to perform rolling circle amplification of the template circular nucleic acid via the DNA polymerase, thereby forming a replication product that comprises concatemeric replications of the target sequence and the backbone sequence joined together and contacting the replication product with an enzyme, thereby producing the replicated circular DNA construct or the replicated linear RNA construct from the replication product. When the one or more enzyme recognition sites comprise recombination sites that flank each end of the target sequence and the enzyme is a recombinase, the contacting results in recombination of the replication product, which removes the backbone sequence from the replication product and joins the ends of the target sequence, thereby producing the replicated circular DNA construct and when the one or more enzyme recognition sites comprise one or more restriction sites flanking one or more ends of the target sequence and the enzyme is a restriction enzyme that cleaves the replication product at the one or more restriction sites, the contacting results in formation of a restriction enzyme-digested replication product. The method further comprises contacting the restriction enzyme-digested replication product with an RNA polymerase configured to perform in vitro transcription, thereby producing the replicated linear RNA construct. In some embodiments, the method further comprises contacting the replicated circular DNA construct with a composition that comprises one or more of a second DNA polymerase, a DNA ligase, a restriction enzyme, or a gyrase, where the contacting is in an amount and duration sufficient to repair nicks in the replicated circular DNA construct and introduce supercoiling in the replicated circular DNA construct. In some embodiments, the step contacting the replicated circular DNA construct with a composition that comprises gyrase is performed after the step of contacting the replicated circular DNA construct with a composition that comprises one or more of a second DNA polymerase, a DNA ligase, or a restriction enzyme.

In some embodiments, the DNA polymerase is a Bst polymerase. In some embodiments, the DNA polymerase is a φ29 polymerase. In some embodiments, the DNA polymerase is a Vent exo-DNA polymerase. In some embodiments, the method further comprises contacting the replication product with an exonuclease after contacting the replication product with the recombinase, wherein the exonuclease selectively digests the cleaved backbone sequence. In some embodiments, the method further comprises purifying the replicated circular DNA construct, thereby removing the backbone sequence. In some embodiments, the one or more enzyme recognition sites comprise one or more restriction sites that flank one or more ends of the target sequence, and wherein the enzyme is the restriction enzyme, thereby producing a replicated linear RNA construct. In some embodiments, the one or more enzyme recognition sites comprise one or more restriction sites that flank one or more ends of the target sequence, and wherein the enzyme is the restriction enzyme, thereby producing a replicated linear DNA construct. In some embodiments, the template circular nucleic acid sequence further comprises a Poly-A tail sequence adjacent to the 3′ end of the target sequence. In some embodiments, the one or more restriction sites are present near the Poly-A tail sequence. In some embodiments, the template circular nucleic acid sequence further comprises a promoter configured to interface with the RNA polymerase. In some embodiments, the RNA polymerase is a T7 RNA polymerase, and wherein the promoter is a T7 promoter.

Another aspect of the invention features an in vitro method of producing a supercoiled replicated circular DNA construct from a template circular nucleic acid construct in a cell-free system, where the template circular nucleic acid construct comprises a target sequence, where the target sequence is configured to form the circular DNA construct upon recombination, recombination sites that flank each end of the target sequence, and a backbone sequence. The method comprising contacting the template circular nucleic acid construct with a Bst, φ29, or Vent exo-DNA polymerase and performing rolling circle amplification of the template circular nucleic acid via the Bst, φ29, or Vent exo-DNA polymerase, thereby forming a replication product that comprises concatemeric replications of the target sequence and the backbone sequence joined together; contacting the replication product with a recombinase, resulting in recombination of the replication product, thereby removing the backbone sequence from the replication product, joining the ends of the target sequence, and producing a replicated circular DNA construct; contacting the backbone sequence with an exonuclease, where the exonuclease selectively digests the cleaved backbone sequence; purifying the replicated circular DNA construct, thereby removing the backbone sequence; and contacting the purified replicated circular DNA construct with a composition that comprises one or more of the following: a DNA polymerase, a DNA ligase, and a gyrase, thereby repairing nicks in the replicated circular DNA construct and producing the supercoiled replicated circular DNA construct.

In some embodiments of any of the preceding methods, the recombinase is a Cre recombinase, an FLP recombinase, a lambda phage integrase, a TP901-1 lactococcal phage recombinase, a Bxb1 integrase, an R4 integrase, a Xer recombinase, a Dre recombinase, or a φC31 integrase. In some embodiments, the recombinase is a Cre recombinase. In some embodiments, the recombinase is a FLP recombinase. In some embodiments, the recombination sites comprise a LoxP sequence or functional variant thereof. In some embodiments, the recombination sites comprise a Lox66 sequence, a Lox71 sequence, or functional variant thereof. In some embodiments, the recombination sites comprise a FRT sequence or functional variant thereof. In some embodiments, the method further comprises contacting the replication product with a restriction enzyme that selectively cleaves the backbone sequence prior to contacting the replication product with the recombinase, thereby forming a digested replication product for contacting with the recombinase.

One aspect of the invention features a replicated circular DNA construct formed by any of the preceding methods.

Another aspect of the invention features a replicated linear RNA construct formed by any of the preceding methods.

In yet another aspect of the invention features a composition comprising the replicated circular DNA construct or the replicated linear RNA construct of any preceding claim and an excipient, diluent, or carrier. In some embodiments, the composition comprises a diluent, wherein the diluent is a storage buffer.

One aspect of the invention features a kit for replicating a circular DNA construct in a cell-free system comprising a template circular nucleic acid construct that comprises a target sequence flanked by recombination sites on each end, wherein the target sequence is configured to form the circular nucleic acid construct upon recombination, and a backbone sequence, a φ29, a Bst, or a Vent exo-DNA polymerase, and a recombinase.

In some embodiments, the kit further comprises one or more of an exonuclease, a DNA polymerase, a DNA ligase, a restriction enzyme, and a gyrase. In some embodiments, the recombinase is a Cre recombinase, an FLP recombinase, a lambda phage integrase, a TP901-1 lactococcal phage recombinase, a Bxb1 integrase, an R4 integrase, a Xer recombinase, a Dre recombinase, or a φC31 integrase.

One aspect of the invention features a kit for replicating a linear RNA construct in a cell-free system comprising a template circular nucleic acid construct that comprises a target sequence, a T7 promoter adjacent to the 5′ end of the target sequence, and a Poly-A tail adjacent to the 3′ end of the target sequence, where the Poly-A tail comprises one or more restriction enzyme sites, a φ29, a Bst, or a Vent exo-DNA polymerase, a restriction enzyme configured to cleave the template circular nucleic acid construct at the one or more restriction enzyme sites, and a T7 RNA polymerase.

Another aspect of the invention features a cell-free method of producing a replicated circular DNA construct from a template circular nucleic acid construct in a cell-free system, where the template circular nucleic acid construct comprises a target sequence, where the target sequence is configured to form the circular DNA construct upon recombination, recombination sites that flank each end of the target sequence, and a backbone sequence. The method comprising contacting the template circular nucleic acid construct with a DNA polymerase configured to perform rolling circle amplification of the template circular nucleic acid via the DNA polymerase, thereby forming a replication product that comprises concatemeric replications of the target sequence and the backbone sequence joined together, contacting the replication product with a recombinase, resulting in recombination of the replication product, thereby removing the backbone sequence from the replication product, joining the ends of the target sequence, and producing a replicated circular DNA construct, purifying the replicated circular DNA construct, where the purified replicated circular DNA construct produced by the cell-free system comprises less endotoxin than a comparable amount of replicated circular DNA construct produced in E. coli, as measured by an amebocyte lysate assay.

In some embodiments, the template circular nucleic acid construct is a single-stranded template circular DNA construct. In some embodiments, the method further comprises producing the single-stranded template circular DNA construct from a template linear DNA construct prior to contacting the template circular nucleic acid construct with a DNA polymerase. In some embodiments, the template linear DNA construct comprises a telRL sequence on each side of the target sequence, and wherein the single-stranded template circular DNA construct is produced from the template linear DNA construct prior to contacting the template circular nucleic acid construct with a DNA polymerase by contacting the template linear DNA construct with a telN protelomerase. In some embodiments, the template circular nucleic acid construct is a double-stranded template circular DNA construct. In some embodiments, the method further comprises producing the double-stranded template circular DNA construct from a template linear DNA construct prior to contacting the template circular nucleic acid construct with a DNA polymerase. In some embodiments, the double-stranded template circular DNA construct is produced from the template linear DNA construct prior to contacting the template circular nucleic acid construct with a DNA polymerase by ligation of the ends of the template linear DNA construct. In some embodiments, the double-stranded template circular DNA construct is produced from the template linear DNA construct prior to contacting the template circular nucleic acid construct with a DNA polymerase by Gibson or GoldenGate assembly.

One aspect of the invention features An in vitro method of producing a replicated linear RNA construct from a template circular nucleic acid construct in a cell-free system, where the template circular nucleic acid construct comprises a target sequence, a T7 promoter adjacent to the 5′ end of the target sequence, and a Poly-A tail adjacent to the 3′ end of the target sequence, where the Poly-A tail is adjacent to one or more restriction enzyme sites. The method comprising contacting the template circular nucleic acid construct with a DNA polymerase configured to perform rolling circle amplification of the template circular nucleic acid via the DNA polymerase, thereby forming a replication product that comprises concatemeric replications of the target sequence, contacting the replication product with restriction enzyme configured to cleave the template circular nucleic acid construct at the one or more restriction enzyme sites, thereby forming a digested replication product, purifying the digested replication product, and contacting the digested replication product with an RNA polymerase, thereby performing in vitro transcription of the purified digested replication product and producing the replicated RNA construct.

In some embodiments, the template circular nucleic acid construct is a single-stranded template circular DNA construct. In some embodiments, the method further comprises producing the single-stranded template circular DNA construct from a template linear DNA construct prior to contacting the template circular nucleic acid construct with a DNA polymerase. In some embodiments, the template linear DNA construct comprises a telRL sequence on each side of the target sequence, and wherein the single-stranded template circular DNA construct is produced from the template linear DNA construct prior to contacting the template circular nucleic acid construct with a DNA polymerase by contacting the template linear DNA construct with a telN protelomerase. In some embodiments, the template circular nucleic acid construct is a double-stranded template circular DNA construct. In some embodiments, the method further comprises producing the double-stranded template circular DNA construct from a template linear DNA construct prior to contacting the template circular nucleic acid construct with a DNA polymerase. In some embodiments, the double-stranded template circular DNA construct is produced from the template linear DNA construct prior to contacting the template circular nucleic acid construct with a DNA polymerase by ligation of the ends of the template linear DNA construct. In some embodiments, the double-stranded template circular DNA construct is produced from the template linear DNA construct prior to contacting the template circular nucleic acid construct with a DNA polymerase by ligation of the ends of the template linear DNA construct. In some embodiments, the double-stranded template circular DNA construct is produced from the template linear DNA construct prior to contacting the template circular nucleic acid construct with a DNA polymerase by Gibson or GoldenGate assembly.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the present disclosure are set forth with particularity in the appended claims. A better understanding of the features and advantages of the present disclosure can be obtained by reference to the following detailed description that sets forth illustrative embodiments, in which the principles of the disclosure are utilized, and the accompanying drawings described below.

FIG. 1 is a schematic illustrating an exemplary template circular nucleic acid construct for cell-free replication having recombination sites recognized by a recombinase (e.g., LoxP or modified LoxP sites). The desired target nucleic acid is replicated and separated from the plasmid backbone using the cell-free methods described herein.

FIG. 2 is a schematic illustrating an exemplary method provided herein for cell-free replication of a circular nucleic acid construct from the exemplary template circular nucleic acid construct of FIG. 1.

FIG. 3 is a schematic illustrating an exemplary template linear nucleic acid construct for replication of an RNA construct.

FIG. 4 depicts methods of producing a template circular DNA construct from a template linear DNA construct.

FIG. 5 depicts an exemplary method provided herein for cell-free replication of an RNA construct from a template circular DNA construct or linear DNA with covalently closed ends.

FIG. 6 depicts an agarose gel (1% agarose) of various DNA samples.

FIG. 7 depicts an agarose gel (1% agarose) of various DNA samples.

FIG. 8 depicts an agarose gel (1% agarose) of various DNA samples.

### DETAILED DESCRIPTION

It is to be understood that both the foregoing general description and the following detailed description are exemplary and explanatory only and are not to be construed as restrictive of the disclosure.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

Disclosed herein are methods and kits for cell-free replication of nucleic acid constructs. The methods and kits can generate replicated nucleic acid molecules devoid of any extraneous backbone sequences required for bacterial replication (e.g., antibiotic resistance genes or origins-of-replication). The methods and kits use a small number of enzymes, making the process much more efficient, scalable, and inexpensive compared to other technologies.

Plasmids are DNA molecules with the capability to replicate autonomously inside a bacterial host. They contain DNA elements that enable them to utilize the host's replication machinery to effect their own propagation. Some plasmids even encode their own replication machinery, and often contain additional accessory genes (such as antibiotic resistance genes) that confer a benefit to their host organism.

Since the development of recombinant DNA technology, plasmid DNA has become a key reagent for all biotechnology research, being the main vehicle for building, propagating, and utilizing DNA in research fields as diverse as biofuels, genetically engineered animals, bio-based industrial chemicals, flavors and fragrances, and biopharmaceutical production.

For some applications such as, for example, cell and gene therapy, nucleic acid-based vaccines, and RNA therapeutics, plasmid DNA is a critical component required in high quantities and is manufactured according to exacting quality control specifications. Often, plasmid DNA for these applications is manufactured at scale through the use of microbial fermentation. Such cell-based replication generally utilize bacterial strains (e.g., E. coli) harboring the plasmid of interest, which are often grown in industrial fermentation bioreactors to achieve high cell densities. Following fermentation, these cells are harvested and lysed to release the plasmid DNA. Through a series of purification steps, the desired plasmid DNA is separated from other molecules present in the lysate. Thus, the desired plasmid DNA is of high-purity.

One application of plasmid DNA is production of RNA, which can be utilizes for RNA-based therapeutics such as mRNA vaccines. RNA production requires large amounts of plasmid DNA to serve as the template for in vitro transcription (IVT) reactions. In the IVT reaction the DNA serves as a template for an RNA polymerase (typically T7 or derivatives), driving the production of RNA in an enzymatic reaction. The most common approach to generating the DNA template is to clone the desired RNA construct (in some embodiments, containing at a minimum a promoter, untranslated regions (UTR) comprising a poly-A tail, and the open reading frame (ORF) for the vaccine or therapeutic protein encoded by the RNA) into a plasmid DNA backbone, introducing the cloned plasmid DNA into a bacterial host, and utilizing the bacterial host as a bioproduction vehicle to generate more copies of the DNA. The host is cultivated in bioreactors to achieve high cell density, and then the plasmid is isolated from the organism through a series of lysis and purification steps. After purification, the plasmid DNA is linearized by a restriction enzyme, and used as a template in the IVT reaction.

There are a number of challenges faced during the production process of plasmid DNA using cell-based replication, some of which are sequence dependent. For example, some plasmid sequences are toxic to the production host organism, preventing their production. Some plasmid sequences are unstable during replication in the host organism, which results in genetic rearrangements, truncations, or other undesired genetic modifications to the plasmid DNA. Some plasmid sequences replicate only at low copy numbers, which results in low plasmid yields.

Further, there are a number of challenges faced during the production process of plasmid DNA that apply generally to microbial production of plasmid DNA. Even with high-copy plasmids, plasmid DNA makes up a very small fraction of the biomass produced during fermentation, meaning that the majority of molecules in the cell lysate are not the desired plasmid DNA. This results in a “needle-in-the-haystack” problem for the purification process, where the non-plasmid molecules need to be removed. Other nucleic acids from the host organism (such as the host's own genome or messenger RNAs) are difficult to purify away from the desired plasmid, resulting in host-related impurities that can be problematic for downstream applications.

In order to replicate in a host organism, the plasmid requires certain extraneous sequences (such as the origin of replication and antibiotic resistance genes) to participate in the DNA replication process. These sequences can pose safety concerns (e.g., immunogenicity) for downstream therapeutic applications. Antibiotics such as kanamycin are often used to maintain plasmids in the host organism. Residual antibiotics in the plasmid DNA preparations can pose health risks for patients in downstream therapeutic applications. Plasmid-producing bacteria such as E. coli contain molecules known as endotoxins (e.g., lipopolysaccharide (LPS)) that are highly toxic to humans. These molecules are also difficult to separate from plasmid DNA, resulting in the need for complex purification techniques and associated analytical tools to confirm their removal.

Accordingly, alternatives to microbial fermentation for plasmid DNA production and RNA production are needed. As such, the present disclosure describes cell-free methods of producing replicated circular DNA constructs (e.g. plasmids) and replicated linear RNA constructs that avoid the issues associated with cell-based replication. The method utilizes a rolling circle amplification of a template circular nucleic acid construct to produce a replication product, which can then be utilized to produce either circular DNA or linear RNA in a cell-free manner.

### Definitions

Unless otherwise indicated, all technical terms used herein have the same meaning as commonly understood by one of ordinary skill in the art. Unless otherwise indicated or obvious from context, the following terms have the following meanings:

As used in this specification and the appended claims, the singular forms “a,” “an,” and “the” include plural references unless the context clearly dictates otherwise. For example, the term “a sample” includes a plurality of samples, including mixtures thereof.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term “comprise” and its grammatical equivalents specifies the presence of stated features, integers, steps, operations, elements, and/or components, but does not preclude the presence or addition of one or more other features, integers, steps, operations, elements, components, and/or groups thereof.

As used herein, the term “about” in reference to a number or range of numbers is understood to mean the stated number and numbers up to +/−10% thereof, (e.g., 10% below the lower listed limit and 10% above the higher listed limit) for the values listed for a range.

The terms “% identical,” “% identity,” and “percent identity,” or grammatical equivalents thereof, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, fifteen-fold, etc.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectable activity (at most, an insignificant amount, e.g., less than about 10%, 5%, 1%, 0.1% or even 0.005%).

The term “protein”, “peptide” and “polypeptide” are used interchangeably to refer to an oligomer of two or more linked amino acids or derivatives of the same.

The terms “complementary” and “complementarity,” as used herein, with reference to a nucleic acid molecule or nucleotide sequence, refer to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term “length” as it applies to a nucleic acid (polynucleotide) or polypeptide may be expressed as “kilobases” (kb) or “base pairs (bp),” and may be used interchangeably with the term, “linked nucleosides.” Thus, a length of 1 kb refers to a length of 1000 linked nucleosides, and a length of 500 bp refers to a length of 500 linked nucleosides. Similarly, a protein having a length of 500 linked amino acids may also be simply described as having a length of 500 amino acids.

The terms “non-naturally occurring,” “engineered,” “genetically modified,” “genetic modification,” and grammatical equivalents as used herein, are used interchangeably and indicate the involvement of the hand of man. The terms, when referring to a cell or expression system, refer to a cell or expression system that is substantially free from at least one other feature with which it is naturally associated in nature, and/or contains a modification (e.g., gene edit, chemical modification, nucleotide sequence, or amino acid sequence) that is not present in the naturally occurring cell or expression system.

The term “biomolecule” as used herein, refers to a polypeptide or polynucleotide that is expressed by a cell or expression system of the invention as described herein. The biomolecule, for example, can be a therapeutic polypeptide or polynucleotide used to alleviate or lessen a symptom of a disease or condition or used for a health, nutritional, or cosmetic benefit in a subject. Examples are, but not limited to, antibodies, messenger RNAs, antisense oligonucleotides, and short interfering RNAs.

The term “circular DNA construct” or “circular nucleic acid construct” includes nucleic acid constructs that are capable of being replicated by rolling circle amplification. For example, non-limiting examples include, but are not limited to circular single-stranded nucleic acid constructs, circular double-stranded nucleic acid constructs, and linear nucleic acid constructs with covalently closed ends.

The term “target sequence” as used herein refers to the sequence that, when processed by the methods described herein, forms the replicated nucleic acid of interest.

### Overview of Nucleic Acid Replication Methods of the Present Disclosure

Disclosed herein are in vitro methods of replicating a nucleic acid construct (such as a circular DNA construct or an RNA construct) from a template nucleic acid construct. The methods described herein can utilize host cells for propagation of template nucleic acid constructs, or can be entirely cell free.

In some aspects, a circular DNA construct or linear DNA with covalently closed ends can be replicated from a template circular nucleic acid construct in a cell-free system, where the template circular nucleic acid construct includes: a target sequence flanked by recombination sites on each end; and a backbone sequence, where the target sequence is configured to form the circular nucleic acid construct upon recombination. In some aspects, this method includes contacting the template circular nucleic acid construct with a polymerase and performing rolling circle amplification of the template circular nucleic acid via the polymerase, thereby forming a replication product that comprises concatemeric replications of the target sequence and the backbone sequence joined together. The method further includes contacting the replication product with a recombinase, resulting in recombination of the replication product, thereby removing the backbone sequence from the replication product, joining the ends of the target sequence, and producing the replicated circular nucleic acid construct from the replication product.

In some aspects, a circular DNA construct, linear DNA with covalently closed ends, or an RNA construct can each be replicated from the same template circular nucleic acid construct in a cell-free system, where the template circular nucleic acid construct includes: a target sequence flanked by both restriction enzyme sites and recombination sites; and a backbone sequence. The circular DNA construct, linear DNA with covalently closed ends, and RNA construct can be replicated by contacting the template circular nucleic acid construct with a polymerase and performing rolling circle amplification of the template circular nucleic acid via the polymerase, thereby forming a replication product that comprises concatemeric replications of the target sequence and backbone sequence. The circular DNA or linear DNA with covalently closed ends construct can then be prepared from the replication product by contacting the replication product with a recombinase, resulting in recombination of the replication product, thereby removing the backbone sequence from the replication product, joining the ends of the target sequence, and producing the replicated circular DNA construct or linear DNA with covalently closed ends from the replication product. Alternatively, the RNA construct can be prepared from the replication product by contacting the replication product with a restriction enzyme that recognizes the restriction enzyme sites at the 3′ end of the target sequence, resulting in restriction digestion of the replication product at the restriction enzyme sites, followed by treatment of the digested replication product with an RNA polymerase, thereby performing in vitro transcription of the digested replication product and producing the replicated RNA construct.

In some aspects, a circular DNA construct can be replicated from a template linear DNA construct (for example, a synthetic linear DNA construct) in a replication system that: (a) produces a template circular DNA construct from the template linear DNA construct, where the template linear DNA construct and template circular DNA construct include a target sequence flanked by recombination sites and a backbone sequence; and (b) replicates the circular DNA construct from the template circular DNA construct as described herein. Once the template circular DNA construct is produced from the template linear DNA construct, the template circular DNA construct is contacted with a polymerase that performs rolling circle amplification of the template circular DNA construct, thereby forming a replication product that comprises concatemeric replications of the target sequence and backbone sequence. The replication product is then contacted with a recombinase, resulting in recombination of the replication product at the recombinase sites, which removes separates the replicated backbone sequence from the replicated target sequence and circularizes the replicated target sequence, thereby producing the replicated circular DNA construct.

In some aspects, an RNA construct can be replicated from a template linear DNA construct (for example, a synthetic linear DNA construct) in a replication system that: (a) produces a template circular DNA construct from the template linear DNA construct, where the template linear DNA construct and template circular DNA construct include a target sequence flanked by a restriction enzyme site at the 3′ end of the gene; and (b) replicates the RNA construct from the template circular DNA construct after linearization by restriction enzyme. Once the template circular DNA construct is produced from the template linear DNA construct, the template circular DNA construct is contacted with a polymerase that performs rolling circle amplification of the template circular DNA construct, thereby forming a replication product that comprises concatemeric replications of the target sequence. The replication product is then contacted with a restriction enzyme, resulting in restriction digestion of the replication product at the restriction enzyme sites, followed by treatment of the digested replication product with an RNA polymerase, thereby performing in vitro transcription of the digested replication product and producing the replicated RNA construct.

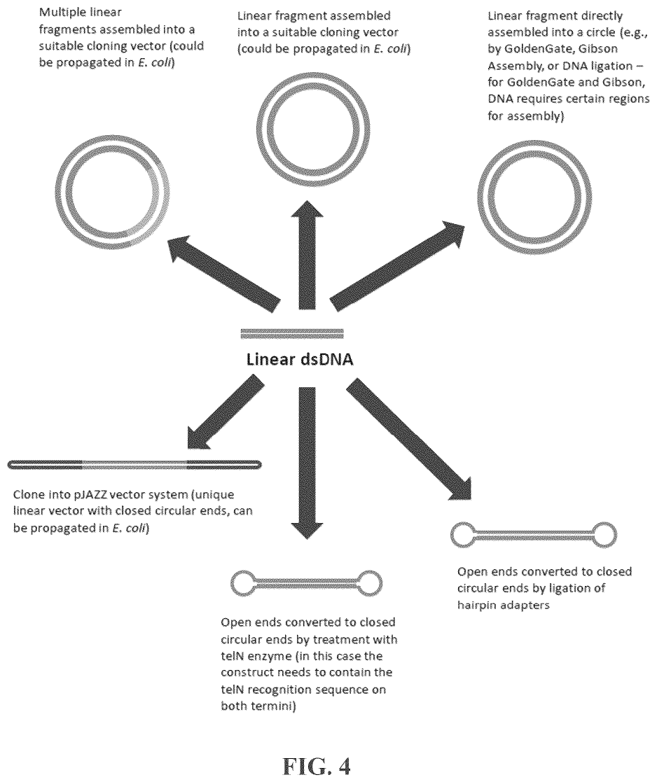

FIG. 4 depicts exemplary methods for producing a template circular DNA construct from a template linear DNA construct. As disclosed herein, the template circular DNA construct can be produced from the template linear DNA construct using a variety of synthetic or molecular cloning techniques). In one example, the template circular DNA construct can be a double-stranded template linear DNA construct with covalently closed ends produced in a host cell from the template linear DNA construct by: (1) cloning the template linear DNA into an expression vector (e.g., pJazz) that propagates the template linear DNA construct in the host cell as a double-stranded linear fragment with covalently closed ends; and (2) transforming the cloned construct into the host cell to produce the double-stranded template linear DNA construct with covalently closed ends. In another example, the template circular DNA construct can be a double-stranded template circular DNA construct produced in a host cell from the template linear DNA construct by: (1) cloning the template linear DNA into an expression vector that propagates the template linear DNA construct in the host cell as double-stranded circular DNA; and (2) transforming the cloned construct into the host cell to produce the double-stranded template circular DNA construct.

Alternatively, the template linear DNA construct with covalently closed ends can be produced from the template linear DNA construct using a cell-free system. Examples of cell-free systems that produce a single stranded template circular DNA construct from the template linear DNA construct include: (a) utilizing a protelomerase (such as telN) to enzymatically catalyzing the formation of closed circular ends by including a telRL sequence on both sides of the target sequence; or (b) ligating or assembling short synthetic hairpin DNA sequences onto the end of the template linear DNA construct, resulting in a linear DNA with covalently closed ends that functions as a template circular DNA construct. Examples of cell-free systems that produce a double-stranded template circular DNA construct from the template linear DNA construct include: (a) assembling the template linear construct with other linear DNA constructs, which can be utilized to produce longer DNA templates for circularization than typical DNA synthesis methods and can be utilized to attach difficult-to-clone segments to the template DNA (such as poly-A tails); (b) assembling the template linear construct directly with itself, converting the double-stranded, linear DNA into a double-stranded circular DNA, which can be accomplished by ligating the termini together with a ligase, or by assembling the termini together with assembly techniques such as GoldenGate or Gibson Assembly.

In some cases, the methods of replicating a nucleic acid construct (such as a circular DNA construct or an RNA construct) from a template nucleic acid construct according to the present disclosure results in high purity nucleic acid products. The methods described herein can increase volumetric yields, mitigate recombination issues with recombination-prone plasmids, improve product quality, and reduce the number of concatemers in the final product.

### Template Nucleic Acid Constructs

Provided herein are template circular nucleic acid constructs, as well as methods of using said template circular nucleic acid constructs for cell-free replication of nucleic acid constructs, and kits for cell-free replication of nucleic acid constructs using said template circular nucleic acid constructs. The template circular nucleic acid constructs as described herein are designed to produce a nucleic acid construct of interest (e.g., circular DNA, linear DNA, circular RNA and/or linear RNA) upon replication by performing discrete manipulations of the template circular nucleic acid construct in a cell-free system. This cell-free system produces a purified replicated nucleic acid construct in usable form (e.g., super coiled circular DNA and/or linear RNA having a Poly-A tail), without the need for a host cell. Utilizing the cell-free replication of the present disclosure provides advantages relative to replication using a host cell. For example, there is no need for antibiotic selection in order to purify the replicated circular nucleic acid, thus reducing safety issues associated with large scale antibiotic use. Additionally, the use of the cell-free system of the present disclosure eliminates host cell-mediated issues with replication, such as reduced replication due to toxicity of the template circular nucleic acid and spurious recombination of the template circular nucleic acid. Further, the cell-free system of the present disclosure can be utilized without the need for a particular origin of replication on the template circular nucleic acid construct, which increases the versatility of the method of the present disclosure relative to other methods of replication that require an origin of replication. Moreover, the use of the cell-free system of the present disclosure can result in higher purity nucleic acid products as compared to nucleic acid products retrieved from a host cell.

As described herein, the template circular nucleic acid can be utilized to produce circular DNA constructs, linear DNA with covalently closed ends, and linear RNA constructs through specific enzymatic manipulations. In order to facilitate these enzymatic manipulations, one or more enzyme recognition sites are provided in the template circular nucleic acid. The specific arrangement and identity of the enzyme recognition sites differ depending on whether circular DNA, circular RNA, linear DNA or linear RNA is going to be produced from the template circular nucleic acid. However, the template circular nucleic acid can include all necessary enzyme recognition sites in the same template circular nucleic acid to produce both circular DNA, circular RNA, linear DNA and linear RNA. Thus, a single template circular nucleic acid can be replicated using rolling circle amplification to produce a replication product, which can then be processed using the respective enzymatic manipulations to produce either circular DNA, circular RNA, linear DNA, or linear RNA.

### Template Nucleic Acid Constructs for Production of Circular DNA

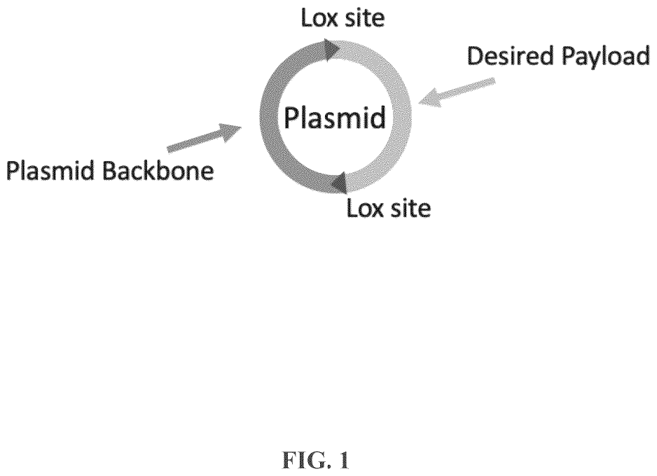

In some embodiments, the template circular nucleic acid constructs can be utilized to produce a replicated circular DNA construct or a linear DNA with covalently closed ends as described herein. Such constructs for production of replicated circular DNA constructs comprise a backbone nucleic acid sequence, a target sequence, and enzymatic sites recognized by enzymes that perform the specific manipulations to produce the replicated product (see FIG. 1). In some embodiments, the backbone sequence optionally comprises an origin of replication and/or an antibiotic resistance gene. In some embodiments, the backbone sequence does not comprise an origin of replication or an antibiotic resistance gene. After replication, the backbone sequence is removed leaving only the target sequence.

In some embodiments, the target sequence is provided in the same construct as the backbone sequence in the template circular nucleic acid for production of replicated circular nucleic acid. The methods described herein replicate the target sequence through rolling circle amplification of the template circular nucleic acid, thus replicating both the target sequence and the backbone sequence. Recombination using a recombinase described herein separates the replicated target sequence from the replicated backbone sequence, which results in conversion of the target sequence into the desired circular nucleic acid.

In some instances, the circular nucleic acid comprises a therapeutic sequence. In some embodiments, the therapeutic sequence can encode a therapeutic (e.g., a therapeutic protein). In some embodiments, the therapeutic sequence itself is the therapeutic (e.g., nucleic acid based vaccines). In some embodiments, the circular nucleic acid is formulated into a composition or pharmaceutical composition. In some embodiments, the circular nucleic acid, composition, or pharmaceutical composition is administered to a subject in need thereof.

In some embodiments, the circular nucleic acid is a commercial expression plasmid. One advantage of the method described herein is the ability to replicate commercial expression plasmids at commercial scale without the use of host cells, thus reducing costs and time associated with cell fermentation and purification. Further, because the method of replication described herein does not require a particular origin of replication, a wider range of commercial plasmids can be replicated without having to adapt the plasmid to a particular expression cell line (e.g., without having to clone origins of replications that are compatible with the host cell for replication.

### Template Nucleic Acid Constructs for Production of Linear DNA, Linear RNA and Circular RNA

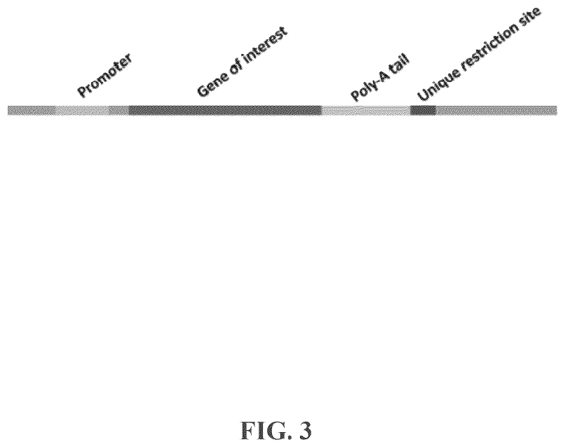

In some embodiments, the template circular nucleic acid constructs can be utilized to produce a replicated linear DNA, linear RNA, and circular RNA construct as described herein. Such template circular nucleic acid constructs for production of replicated linear DNA, linear RNA, and circular RNA constructs comprise one or more of: a target sequence; a promoter adjacent to the 5′ end of the target sequence, a Poly-A tail adjacent to the 3′ end of the target sequence, and one or more restriction enzyme sites. In some embodiments, template circular nucleic acid constructs for production of replicated linear RNA constructs can be produced from template linear DNA constructs, which can be circularized by methods described herein prior to rolling circle amplification. FIG. 3 depicts a schematic of an exemplary template linear DNA construct for production of a replicated RNA construct. As shown in FIG. 3, the method as described herein produces a linear DNA intermediate that is reverse transcribed into linear RNA. In some embodiments, the linear DNA intermediate can be isolated and purified directly as the final product.

In some embodiments, the linear RNA products produced as shown in FIG. 3 can be circularized in vitro to produce circularized RNA products. In some embodiments, replicated RNA constructs produced according to methods herein can be circularized using chemical or enzymatic approaches. In some embodiments, replicated RNA constructs can be circularized using chemical synthesis. In some cases, replicated RNA constructs can be circularized using chemical ligation. In some cases, replicated RNA constructs can be circularized using enzymatic synthesis of linear RNA precursor. Non-limiting examples of circular RNA enzymatic synthesis include in vitro transcription, RNA monophosphorylation, or enzymatic ligation. In some embodiments, enzymatic ligation can be catalyzed by a DNA ligase 1, a T4 RNA ligase 1, a T4 RNA ligase 2, or a hairpin ribozyme (HPR). In some cases, replicated RNA constructs can be circularized using a permuted intron-exon (PIE) system with group I introns, or a PIE system with group II introns. In some embodiments, the replicated RNA constructs can be circularized in vivo. In some cases, replicated RNA constructs resulting from the methods disclosed herein can be circularized using repetitive intronic complementary sequences (ICS), non-repetitive ICS. In some cases, replicated RNA constructs can include elements that promote circular RNA formation. Non-limiting examples of such elements include repetitive Alu elements, non-repetitive regions of reverse complementary sequence, or protein-binding sites for Muscleblind or Quaking.

### Methods of Cell-Free Replication of Circular DNA Constructs

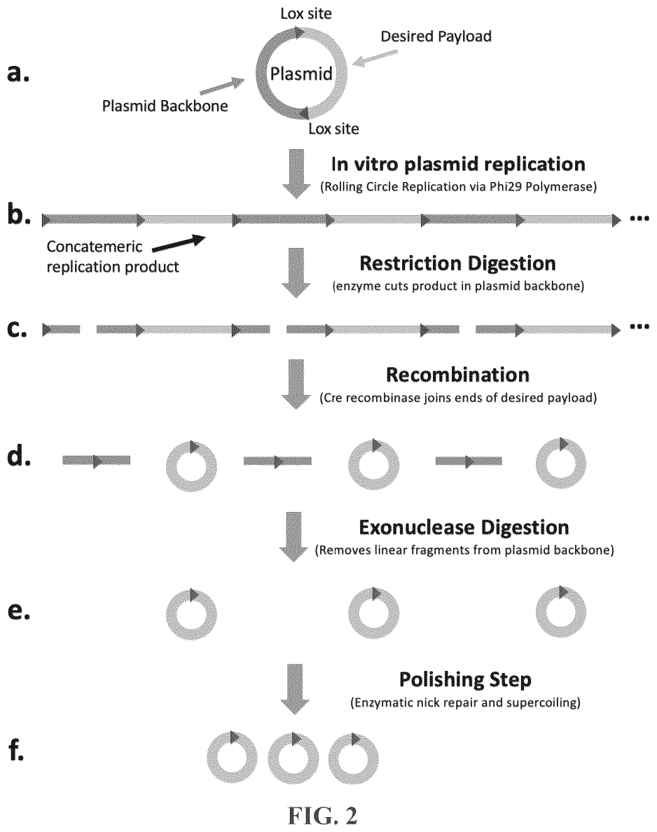

Provided herein are methods for cell-free replication of circular nucleic acid constructs comprising contacting a template circular nucleic acid construct as described herein with a combination of enzymes that result in replication and production of the circular nucleic acid, where the template circular nucleic acid construct comprises a target sequence and a backbone sequence as described herein. FIG. 2 provides an exemplary workflow for producing a replicated circular DNA construct from a template circular nucleic acid construct. The circular nucleic acid of interest is amplified through rolling circle amplification of the template circular nucleic acid construct, which replicates the target sequence and backbone sequence. Rolling circle amplification of the template circular nucleic acid is performed via a polymerase configured to perform rolling circle amplification, thereby forming a replication product that comprises concatemeric replications of the target sequence and the backbone sequence joined together. The replication product is then contacted with a restriction enzyme to cut the concatemeric product into monomeric, linear nucleic acid product. The restriction digested replication product is contacted with a recombinase, resulting in recombination of the replication product, thereby removing the backbone sequence from the replication product, joining the ends of the target sequence, and producing the replicated circular nucleic acid construct from the replication product.

In some embodiments, the template circular nucleic acid construct is initially created (e.g., assembly of nucleic acid sequences) using cell-free techniques. In some embodiments, a vector designed for a particular host cell is chosen and modified to comprise the target sequence and the sites that allow for the enzymatic modifications described herein. In some embodiments, a eukaryotic vector is modified to comprise the target sequence and the enzymatic modification sites. In some embodiments, a bacterial vector is modified to comprise the target sequence and the enzymatic modification sites. In some embodiments, the template circular nucleic acid construct can be initially produced using standard cell-based cloning, selected for using conventional selection techniques, and subsequently purified from the host cell for use in the in vitro cell-free methods described herein. In some embodiments, the template circular nucleic acid construct is replicated without purification or only partial purification from the host cell.

The initial host cell can be from a variety of sources, including but not limited to bacterial, fungal, yeast, animal, insect, mammal, fish, or plant host cells. In some embodiments, the host cell is E. coli. Indeed, because the template circular nucleic acid construct does not require a particular origin of replication for replication in the cell-free system described herein, any origin of replication can be incorporated into the backbone sequence for initial production in a host cell without the need for subsequent removal where the host cell for replication differs from the expression host cell.

In some embodiments, the template circular nucleic acid does not rely on any cell-based replication during construction. Indeed, any known method for producing the template circular nucleic acid can be utilized.

The cell free replication method utilizes one or more of the following steps to produce a replicated, purified circular nucleic acid from a template circular nucleic acid:
contacting the template circular nucleic acid construct having the target sequence flanked by recombination sites and the backbone sequence with a polymerase capable of performing rolling circle amplification of the template circular nucleic acid, thereby forming a replication product that comprises concatemeric replications of the target sequence and the backbone sequence joined together;
contacting the replication product with a restriction enzyme, thereby forming a digested replication product, wherein the restriction enzyme selectively cleaves the backbone sequence;
contacting the replication product with a recombinase, resulting in recombination of the replication product, thereby: removing the backbone sequence from the replication product, joining the ends of the target sequence, and producing a mixture comprising recombined backbone sequence and the circular nucleic acid;
contacting the mixture comprising recombined backbone sequence and the circular nucleic acid with an exonuclease, wherein the exonuclease selectively digests the recombined backbone sequence;
purifying the mixture after contacting with the exonuclease, thereby removing the digested recombined backbone sequence; and
contacting the circular nucleic acid construct with a composition that repairs any nicks that may be present in the circular nucleic acid construct, which can include one or more of: a DNA polymerase, DNA ligase, and a gyrase. Each step is described in greater detail below.

### Rolling Circle Amplification (RCA)

As described herein, the cell-free method of producing replicated circular DNA can include contacting a template circular nucleic acid construct with a DNA polymerase configured to perform rolling circle amplification (RCA) of the template circular nucleic acid via the DNA polymerase. Using RCA, the template circular nucleic acid is replicated, thereby forming a replication product that comprises concatemeric replications of the target sequence and the backbone sequence joined together. RCA is an isothermal nucleic acid amplification technique where the polymerase continuously adds single nucleotides to a primer annealed to a circular template which results in a long concatemer of DNA that contains tens to hundreds of repeats. In general, RCA produces a linear amplification of DNA, as each circular template grows at a given speed for a certain amount of time. For example, RCA can amplify the circular template rapidly without thermal cycling and is very sensitive. RCA further has significant processivity and efficiency, which can result in production of microgram quantities of replication product using only nanogram quantities of template. Further, RCA is capable of synthesizing long strands of DNA greater than 70 kb per binding event and rapidly produces the replicated circular nucleic acids (e.g., a reaction time of about 20 minutes compared to 2 days using conventional techniques).

Any polymerase can be utilized that is capable of RCA in the methods described herein. Furthermore, because RCA does not require a polymerase that specifically recognizes a particular origin of replication, it is unnecessary to add an origin of replication into the template (i.e., in a backbone sequence or target sequence) in order to replicate the template. Thus, utilizing RCA reduces processivity time and labor associated with adding and/or removing a particular origin of replication that is compatible with a given polymerase. Further, the presence of exogenously-added origins of replication in a therapeutic construct can result in increased immunogenicity when the construct is administered to a subject. Thus, the risk of immunogenicity due to the presence of such origins of replication is mitigated using the method described herein. Accordingly, the current method utilizing RCA to replicate a template circular nucleic acid is superior to processes relying on polymerases that require specific origins of replication. Exemplary polymerases that are capable of RCA include φ29, Bst, Vent exo-DNA polymerase, RNA polymerase, T4, T7 Sequenase, Taq, or Klenow. In some embodiments, the polymerase is φ29. RCA can be conducted at a constant temperature (room temperature to 65° C.) in both free solution and on top of immobilized targets (solid phase amplification). In some embodiments, hyperbranched RCA is used, where primers anneal to the original RCA products are added and are extended. In some embodiments, circle to circle is used, where the RCA products are digested with a restriction enzyme and ligated into new circular templates using a restriction oligo, followed by a new round of RCA with a larger amount of circular templates for amplification. In some embodiments, the primer is a random hexamer. In some embodiments, the primer is one or more site-specific replication primers.

### Restriction Enzyme Digestion

As described herein, the cell-free replication method can include digestion of the backbone sequence using a restriction enzyme. A restriction enzyme, restriction endonuclease, REase, ENase or restrictase is an enzyme that cleaves DNA into fragments at or near specific recognition sites within molecules known as restriction sites. Restriction enzymes recognize a specific sequence of nucleotides and produce a double-stranded cut in the DNA.

In some embodiments, restriction enzyme sites are incorporated into the backbone sequence of the template circular nucleic acid construct. Because the restriction enzyme sequence is incorporated into the backbone sequence only, the target sequence (and therefore the circular nucleic acid of interest) is not digested by the restriction enzyme. Thus, restriction enzyme digestion allows for facile processing (e.g., removal) of the backbone sequence.

In some embodiments, a restriction digest using restriction enzymes can be performed on the concatemers formed after replication. Digestion of the concatemers at the backbone sequence allows for a size reduction of the concatemeric replication product, which improves the handling and further processing of the concatemeric replication product.

In some embodiments, a restriction digest can be performed after the target sequence and backbone sequence are separated (e.g., by recombination). Because the restriction sites are included only in the backbone sequence, separated backbone sequence can be digested into smaller nucleic acids without digesting the recombined circular nucleic acid of interest. This can result in partial or complete removal of the backbone sequence, and improves the processivity and efficiency of the subsequent purification of the circular nucleic acid from the backbone sequence.

In some embodiments, the concatemers are at least partially purified prior to restriction enzyme digestion. In some embodiments, a phenol/chloroform extraction with alcohol precipitation, an agarose gel electrophoresis followed by gel extraction, or an agarose gel electrophoresis followed by electroelution. In some embodiments, the concatemers are subjected to a heat treatment to inactivate the restriction enzyme.

Any restriction enzyme can be utilized in the method as described herein, so long as the restriction enzyme site is present in the backbone sequence and is not present in the target sequence. Examples of restriction enzymes that can be utilized in the method described herein include: Alu I, BamH I, Bgl II, Cla I, Dpn I, Dpn II, Eco47 III, EcoR I, EcoR V, Hae III, Hind III, Hpa II, Kpn I, Msp I, Nco I, Nde I, Nhe I, Not I, Pst I, Rsa I, Sac I, Sac II, Sal I, Sau3A I, Sfi I, Sma I, Xba I, Xho I, and Xma I.

In some embodiments, other enzymes or enzyme systems are used to cleave the concatemers. In some embodiments, the other enzymes or enzyme systems are CRISPR systems, zinc finger nucleases, transcription activator-like effector nuclease (TALEN), oligonucleotide directed mutagenesis, site specific nucleases, and meganucleases.

### Recombinases

As described herein, the cell-free replication method can include contacting the concatemeric replicated template with a recombinase, which results in recombination of the concatereric product and separation of the target sequence from the backbone sequence. Site-specific recombination, also known as conservative site-specific recombination, is a type of genetic recombination in which DNA strand exchange takes place between segments possessing at least a certain degree of sequence homology. Enzymes known as site-specific recombinases (SSRs) perform rearrangements of DNA segments by recognizing and binding to short, specific DNA sequences (sites), at which they cleave the DNA backbone, exchange the two DNA helices involved, and rejoin the DNA strands. In some embodiments, the recombinase is a tyrosine recombinase. In some embodiments, the recombinase is a serine recombinase. In some embodiments, the recombinase is a Cre recombinase, an FLP recombinase, a lambda phage integrase, a TP901-1 lactococcal phage recombinase, a Bxb1 integrase, an R4 integrase, a Xer recombinase, a Dre recombinase, or a φC31 integrase. In some embodiments, the recombinase is a Cre recombinase.

Using a recombinase to circularize the target sequence and remove the backbone sequence is superior to other methods that rely on protelomerases. Such methods utilizing protelomerases to circularize the target sequence and remove the backbone sequence produce linear DNA molecules with closed ends (so-called DoggyBone molecules). Such DoggyBone constructs are limited in that they have a very different topology from traditional circular, supercoiled plasmid DNA, and thus the safety and/or efficacy of such constructs is unknown. Because the current method produces traditional circular, supercoiled plasmid DNA as the end product, the current method has a more well understood efficacy and safety profile relative to methods utilizing protelomerases, highlighting the superiority of the current method which utilizes recombinases in lieu of protelomerases.

The use of Cre recombinase in combination with the Lox66 and Lox71 sites (or functional variants thereof) allows for recombination at these recombination sites, resulting in circularization of the target sequence (thereby forming the circular nucleic acid of interest) and splicing of excised backbone sequences without substantial reversion to linear nucleic acid constructs. An exemplary recombination scheme using Cre recombinase in combination with Lox66 and Lox72 recombination sites is provided in Scheme 1 below:

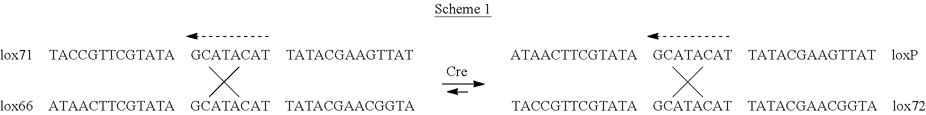

As shown in Scheme 1, the forward reaction is highly preferred as noted by the longer arrow, indicating that the circularized nucleic acid construct after undergoing recombination is quite stable. Indeed, using Cre recombinase in combination with Lox66 and Lox71 recombination sites improves recombination efficiency (relative to, for example using LoxP recombination sites) since the forward reaction in Scheme 1 is largely irreversible. In some embodiments, the backbone sequence is at least partially removed from the replicated circularized nucleic acid construct.

The use of FLP recombinase in combination with FRT sites or functional variant thereof allows for recombination at these recombination sites, resulting in circularization of the target sequence (thereby forming the circular nucleic acid of interest) and splicing of excised backbone sequences without substantial reversion to linear nucleic acid constructs.

The use of TP901-1 lactococcal phage recombinase in combination with TP901-1 attB/attP sites allows for recombination at these recombination sites, resulting in circularization of the target sequence (thereby forming the circular nucleic acid of interest) and splicing of excised backbone sequences without substantial reversion to linear nucleic acid constructs.

### Exonucleases

As described herein, the cell-free replication method can include exonuclease digestion of the backbone sequence after recombination. Exonucleases are enzymes that work by cleaving nucleotides one at a time from the end (exo) of a polynucleotide chain. A hydrolyzing reaction that breaks phosphodiester bonds at either the 3′ or the 5′ end occurs. Because the recombination produces a circularized nucleic acid that lacks a 3′ and 5′ end, the circular nucleic acid is not digested with the exonuclease. However, the exonuclease is able to hydrolyze the excised backbone sequence because the excised backbone sequence is a linear nucleic acid having a 5′ end and a 3′ end.

In some embodiments, the exonuclease is an exonuclease I, II, III, IV, V, VI, VII, or VIII type. In some embodiments, the exonuclease is exonuclease V. In some embodiments, the exonuclease is a plasmid-safe DNase, which has improved activity against linear strands of nucleic acids and decreased activity against circular nucleic acid sequences.

### Ligases

As described herein, the cell-free replication method can include ligation of the circularized nucleic acid to repair any nicks present after the replication. Ligases are versatile and ubiquitous enzymes that join the 3′ hydroxyl and 5′ phosphate ends to form a phosphodiester bond. Ligases have a metal binding site which is capable of recognizing nicks in DNA. The ligase forms a DNA-adenylate complex, assisting recognition. During repair the ligase is adenylylated, transfers the AMP to the nucleic acid construct, and then forms the phosphodiester bond, thereby sealing the nick. In some embodiments, a composition of enzymes are used to repair any nicks in the replicated circular nucleic acid. In some embodiments, the composition comprises one or more of a DNA polymerase, a DNA ligase, and a gyrase. In some embodiments, the composition comprises a DNA polymerase and a DNA ligase. In some embodiments, the composition comprises a DNA polymerase and a gyrase. In some embodiments, the composition comprises a DNA ligase and a gyrase. In some embodiments, the composition comprises a DNA polymerase, a DNA ligase, and a gyrase. In some embodiments, the composition comprises one or more of Taq DNA Ligase, Endonuclease IV, BST DNA Polymerase, Fpg and/or uracil-DNA Glycosylase (UDG), T4 PDG (T4 Endonuclease V) or Endonuclease VIII.

### Gyrases

As described herein, the cell-free replication method can contact the circularized nucleic acid with a DNA gyrase. DNA gyrase, or simply gyrase, is an enzyme within the class of topoisomerase. The enzyme causes negative supercoiling of the DNA or relaxes positive supercoils. The unique ability of gyrase to introduce negative supercoils into DNA at the expense of ATP hydrolysis allows formation of supercoiled circular replicated circular nucleic acid sequences. In some embodiments, the gyrase is a holoenzyme comprising gyrA and gyrB. In some embodiments, the gyrase is combined with the ligation step (nick repair step). In some embodiments, the gyrase is used after the ligation step (nick repair step).

### Methods of Cell-Free Replication of Linear RNA Constructs

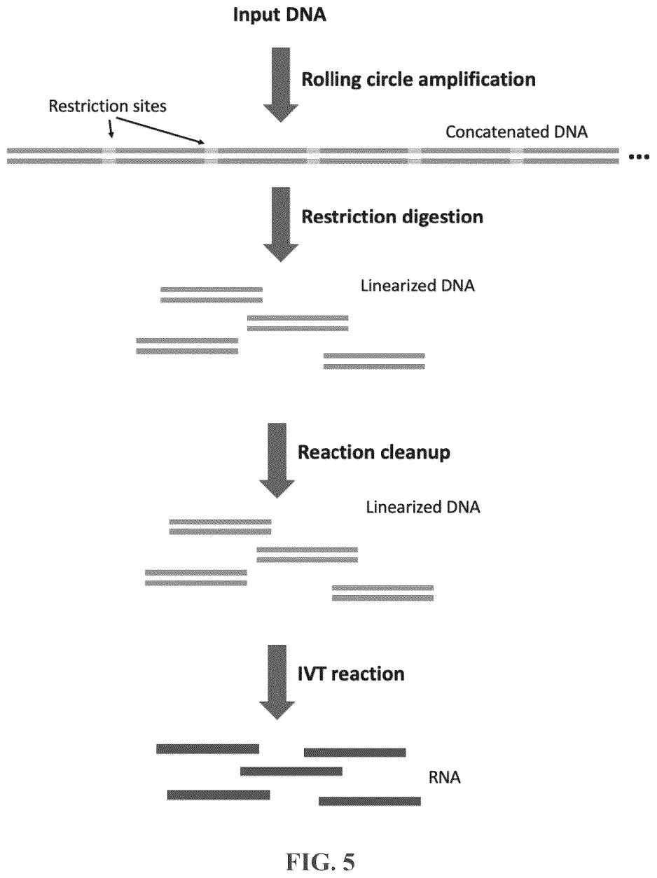

Provided herein are cell-free methods for producing replicated linear RNA from a template circular nucleic acid construct, where the method comprises contacting a template circular nucleic acid construct as described herein with a combination of enzymes that result in replication and production of the linear RNA construct, where the template circular nucleic acid construct comprises one or more of: a target sequence; a promoter adjacent to the 5′ end of the target sequence, a Poly-A tail adjacent to the 3′ end of the target sequence, and one or more restriction enzyme sites. FIG. 5 provides an exemplary workflow for producing a replicated linear RNA construct from a template circular nucleic acid construct. The linear RNA construct (in DNA form) is amplified through rolling circle amplification of the template circular nucleic acid construct as discussed with respect to production of circular DNA via a polymerase configured to perform rolling circle amplification, thereby forming a replication product that comprises concatemeric replications of the target sequence. The replication product is then contacted with a restriction enzyme that site specifically cleaves the replication product at the replication site (e.g., at the Poly-A tail), thereby generating a plurality of digested linear template DNA constructs. The resulting digested linear template DNA construct after purification is utilized as a substrate for in vitro transcription, which results in production of the replicated linear RNA construct.

The template circular nucleic acid construct for production of the linear RNA construct can be constructed using the methods described herein. In some embodiments, the template circular nucleic acid does not rely on any cell-based replication during construction. Indeed, any known method for producing the template circular nucleic acid can be utilized.

The cell-free method for producing replicated linear RNA constructs from a template circular DNA construct method utilizes one or more of the following steps:
contacting the template circular nucleic acid construct with a DNA polymerase configured to perform rolling circle amplification of the template circular nucleic acid via the DNA polymerase, thereby forming a replication product that comprises concatemeric replications of the target sequence;
contacting the replication product with restriction enzyme configured to cleave the template circular nucleic acid construct at the one or more restriction enzyme site near the Poly-A tail, thereby forming a digested replication product;
purifying the digested replication product; and
contacting the digested replication product with an RNA polymerase such as T7 RNA polymerase, thereby performing in vitro transcription of the purified digested replication product and producing the replicated RNA construct. Certain steps are similar or the same as those performed for production of replicated circular DNA. Any differences from those described for production of replicated circular DNA are described below in detail.

### Rolling Circle Amplification (RCA)

As described herein, the cell-free method of producing replicated linear RNA can include contacting a template circular nucleic acid construct with a DNA polymerase configured to perform rolling circle amplification (RCA) of the template circular nucleic acid via the DNA polymerase. Using RCA, the template circular nucleic acid is replicated, thereby forming a replication product that comprises concatemeric replications of the target sequence and other features present in the template nucleic acid construct (e.g., promotor, Poly-A tail, etc.).

Any polymerase that is capable of RCA described above with respect to production of replicated circular DNA constructs can be utilized in the method for production of replicated linear RNA described herein.

### Restriction Enzyme Digestion

As described herein, the cell-free method of producing replicated linear RNA can include contacting a template circular nucleic acid construct replication product with a restriction enzyme configured to cleave the template circular nucleic acid construct at one or more restriction enzyme sites, thereby forming a digested replication product. In some embodiments, one or more restriction enzymes sites are provided in a Poly-A tail that is positioned at the 3′ end of the target sequence. Because the one or more restriction enzyme sites are present on each copy of the replicated cassette (i.e. the target sequence and included features such as the promotor and Poly-A tail) in the concatemeric replication product, contacting the replication product with the restriction enzyme results in separation of each copy of the replicated cassette into individual linear DNA templates that encode the linear RNA construct.

Any restriction enzyme described above with respect to production of replicated circular DNA can be utilized in the method of producing linear RNA described herein, so long as the restriction enzyme site is not present in the target sequence of the replicated cassette. In some embodiments, a single unique restriction enzyme site is provided adjacent to the Poly-A tail, with the corresponding restriction enzyme that cleaves at the single unique restriction enzyme site being utilized in the method of producing linear RNA described herein.

### RNA Polymerases

As described herein, the cell-free method of producing replicated linear RNA can include can in vitro transcription of a linear DNA template via an RNA polymerase, thereby forming linear RNA from the linear DNA template. In some instances, the linear DNA template can be the linear DNA template that is produced by contacting a concatemeric replication product having multiple copies of the DNA template with a restriction enzyme that separates copy into individual linear DNA templates as described herein. In some instances, the restriction enzyme-digested concatemeric replication product can be partially or fully purified prior to the in vitro transcription.

Any RNA polymerase that is capable of in vitro transcription to produce transcribed linear RNA from a linear DNA template can be utilized in the method described herein, such as a T7 RNA polymerase, a T3 RNA polymerase, a SP6 RNA polymerase, or functional variants of any of these. In some embodiments, a T7 RNA polymerase is utilized. In general, the choice of the RNA polymerase utilized will match the corresponding promotor provided in the template nucleic acid. For example, where a T7 promotor is included in the template nucleic acid at a position 5′ to the target sequence for in vitro transcription, a T7 RNA polymerase is utilized for the in vitro transcription.

### Compositions

Provided herein are compositions comprising a cell-free replicated circular DNA construct, linear DNA with covalently closed ends, or replicated linear RNA construct, and an excipient, diluent, or carrier. Also provided herein are compositions comprising one or more enzymes described herein and an excipient, diluent, or carrier.

In some embodiments, methods, compositions, and kits disclosed herein can comprise a mixture containing the circular nucleic acid constructs according to the present disclosure and can take the form of liquid, solid, semi-solid or lyophilized powder forms, such as, for example, solutions, suspensions, and emulsions. In some embodiments, methods, compositions, and kits disclosed herein can comprise a mixture containing one or more enzymes used to generate the cell-free replicated circular nucleic acid construct according to the present disclosure and can take the form of liquid, solid, semi-solid or lyophilized powder forms, such as, for example, solutions, suspensions, and emulsions.

In some embodiments, a composition can include a conventional carrier or excipient and can additionally include other agents, carriers, adjuvants, additives and the like. In some cases, the composition can be about 0.1% to about 85%, about 0.5% to about 75% by weight of a circular nucleic acid construct of the disclosure, with the remainder consisting essentially of suitable excipients.

In some embodiments, a composition herein can comprise an excipient. In some embodiments, a composition herein can comprise one or more of the following excipients: acacia, acesulfame potassium, acetic acid-glacial, acetone, acetyltributyl citrate, acetyltriethyl citrate, adipic acid, agar, albumin, alcohol, alginic acid, aliphatic polyesters, alitame, allantoin, almond oil, alpha hydroxy acids, alpha tocopherol, aluminum hydroxide adjuvant, aluminum monostearate, aluminum oxide, aluminum phosphate adjuvant, ammonia solution, ammonium alginate, ammonium chloride, argan oil, ascorbic acid, ascorbyl glucoside, ascorbyl palmitate, aspartame, attapulgite, azelaic acid, azulene, bakuchiol, beta glucan, beta-hydroxy-acids, bentonite, benzalkonium chloride, benzethonium chloride, benzoic acid, benzyl alcohol, benzyl benzoate, boric acid, bronopol, butylated glycol, butylated hydroxyanisole, butylated hydroxytoluene, butylene glycol, butylparaben, calcium acetate, calcium alginate, calcium carbonate, calcium chloride, calcium hydroxide, calcium lactate, calcium phosphate-dibasic anhydrous, calcium phosphate-dibasic dihydrate, calcium phosphate-tribasic, calcium silicate, calcium stearate, calcium sulfate, canola oil, capric glycol, capric triglyceride, carbomer, carbon dioxide, carboxymethylcellulose calcium, carboxymethylcellulose sodium, carrageenan, castor oil, castor oil-hydrogenated, cellulose-microcrystalline, cellulose-microcrystalline and carboxymethylcellulose sodium, cellulose-powdered, cellulose-silicified microcrystalline, cellulose acetate, cellulose acetate phthalate, ceramides, ceresin, cetostearyl alcohol, cetrimide, cetearyl alcohol, cetyl alcohol, cetylpyridinium chloride, chitosan, chlorhexidine, chlorobutanol, chlorocresol, chlorodifluoroethane (hcfc), chlorofluorocarbons (cfc), chloroxylenol, cholesterol, citric acid monohydrate, coconut oil, collagen, colloidal silicon dioxide, coloring agents, copper peptide, copovidone, corn oil, corn starch and pregelatinized starch, cottonseed oil, cresol, croscarmellose sodium, crospovidone, cyclodextrins, cyclomethicone, denatonium benzoate, desitin, dextrates, dextrin, dextrose, dibutyl phthalate, dibutyl sebacate, diethanolamine, diethyl phthalate, difluoroethane (hfc), dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol stearates, ethylene vinyl acetate, ethylparaben, fatty acids, ferulic acid, fructose, fumaric acid, gelatin, glucose-liquid, glycerin, glycerol, glyceryl behenate, glyceryl monooleate, glyceryl monostearate, glyceryl palmitostearate, glycine, glycofurol, glycolic acid, glycol stearate, guar gum, hectorite, heptafluoropropane (hfc), hexetidine, hydrocarbons (hc), hyaluronic acid, hydrochloric acid, hydrocortisone, hydrophobic colloidal silica, mesoporous silica, hydroquinone, hydroxyethyl cellulose, hydroxyethylmethyl cellulose, hydroxypropyl betadex, hydroxypropyl cellulose, hydroxypropyl cellulose-low-substituted, hydroxypropyl starch, hypromellose, hypromellose acetate succinate, hypromellose phthalate, imidurea, inulin, iron oxides, isomalt, isoparaffin, isopropyl alcohol, isopropyl myristate, isopropyl palmitate, jojoba oil, kaolin, kojic acid, lactic acid, lactitol, lactose-anhydrous, lactose-inhalation, lactose-monohydrate, lactose-monohydrate and corn starch, lactose-monohydrate and microcrystalline cellulose, lactose-monohydrate and povidone, lactose-monohydrate and powdered cellulose, lactose-spray-dried, lanolin, lanolin-hydrous, lanolin alcohols, lauric acid, lecithin, leucine, linoleic acid, macrogol 15 hydroxystearate, magnesium aluminum silicate, magnesium carbonate, magnesium oxide, magnesium silicate, magnesium stearate, magnesium trisilicate, maleic acid, malic acid, maltitol, maltitol solution, maltodextrin, maltol, maltose, mannitol, medium-chain triglycerides, meglumine, menthol, methionine, methylcellulose, methylparaben, mineral oil, mineral oil-light, mineral oil and lanolin alcohols, monoethanolamine, monosodium glutamate, monothioglycerol, myristic acid, myristyl alcohol, neohesperidin dihydrochalcone, neotame, niacinamide, nitrogen, nitrous oxide, octyldodecanol, oleic acid, oleyl alcohol, olive oil, palmitic acid, paraffin, peanut oil, pectin, PEG-8 stearate, pentetic acid, petrolatum, petrolatum and lanolin alcohols, phenol, phenoxyethanol, phenylethyl alcohol, phenylmercuric acetate, phenylmercuric borate, phenylmercuric nitrate, phospholipids, phosphoric acid, phytic acid, phytosphingosine, polacrilin potassium, poloxamer, polycarbophil, polydextrose, poly (dl-lactic acid), polyethylene glycol, polyethylene oxide, polymethacrylates, poly(methyl vinylether/maleic anhydride), polyoxyethylene alkyl ethers, polyoxyethylene castor oil derivatives, polyoxyethylene sorbitan fatty acid esters, polyoxyethylene stearates, polyoxylglycerides, polyparaben, polysorbate 60, polysorbate 80, polyvinyl acetate phthalate, polyvinyl alcohol, potassium alginate, potassium alum, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, propylparaben sodium, pyrrolidone, raffinose, Retin-A, retinol and retinoic acid derivatives, saccharin, saccharin sodium, safflower oil, salicylic acid, saponite, sesame oil, shellac, simethicone, sodium acetate, sodium alginate, sodium ascorbate, sodium benzoate, sodium bicarbonate, sodium borate, sodium carbonate, sodium chloride, sodium citrate dihydrate, sodium cyclamate, sodium formaldehyde sulfoxylate, sodium hyaluronate, sodium hydroxide, sodium lactate, sodium lauryl sulfate, sodium metabisulfite, sodium phosphate-dibasic, sodium phosphate-monobasic, sodium propionate, sodium starch glycolate, sodium stearyl fumarate, sodium ascorbyl phosphate, sodium deoxycholate, sodium hydroxide, sodium lauroyl lactylate, sodium lauryl sulfate, sodium palmitate, sorbitan stearate, sodium sulfite (E221), sprinolactone, sodium sulfite, sodium thiosulfate, sorbic acid, sorbitan esters (sorbitan fatty acid esters), sorbitan monostearate, sorbitol, soybean oil, sphingomyelins, starch, starch-pregelatinized, starch-sterilizable maize, stearic acid, stearyl alcohol, squalene, sucralose, sucrose, sucrose octaacetate, sugar-compressible, sugar-confectioner's, sugar spheres, sulfobutylether b-cyclodextrin, sulfur dioxide, sulfuric acid, sunflower oil, suppository bases-hard fat, tagatose, talc, tartaric acid, tetrafluoroethane (hfc), thaumatin, thimerosal, thymol, titanium dioxide, tragacanth, trehalose, tretinoin, triacetin, tributyl citrate, tricaprylin, triethanolamine, triethyl citrate, triethanolamine, triolein, undecylenic acid, vanillin, vegetable oil-hydrogenated, vitamins, vitamin e polyethylene glycol succinate, water, wax-anionic emulsifying, wax-carnauba, wax-cetyl esters, wax-microcrystalline, wax-nonionic emulsifying, wax-white, wax-yellow, xanthan gum, xylitol, zein, zinc acetate, and/or zinc stearate.

In some cases, a composition can comprise a carrier or a diluent. In some instances, a carrier or diluent can comprise a water, an alcohol, a salt solution (e.g., saline), or a mixture thereof. In some instances, a carrier can comprise a carbohydrate, a buffer, a salt, a pH adjuster, or any combination thereof. In some cases, a composition herein can comprise a buffering agent, a polymer, an antioxidant, a preservative, a chelating agent, a viscomodulator, a tonicifier, a flavorant, a colorant, an odorant, an opacifier, a suspending agent, a binder, a filler, a plasticizer, a lubricant, or any combination thereof.

In some embodiments, a liquid composition can be prepared by dissolving or dispersing the population of circular nucleic acid constructs or plasmids in one or more of the above formulations (about 0.5% to about 20% by weight or more), and optional adjuvants, in a carrier, such as, for example, aqueous saline, aqueous dextrose, glycerol, or ethanol, to form a solution or suspension. In some cases, for use in a liquid preparation, the composition can be prepared as a solution, suspension, or emulsion, being supplied either in liquid form or a dried form suitable for hydration in water or normal saline.

### Kits

Provided herein are kits and containers thereof suitable for performing any one of the methods disclosed herein.

In some embodiments, the kits for replicating a circular nucleic acid construct in a cell-free system comprise one or more of (a) a template circular nucleic acid construct that comprises: (i) a target sequence flanked by recombination sites on each end, wherein the target sequence is configured to form the circular nucleic acid construct upon recombination, and (ii) a backbone sequence, (b) a polymerase configured to perform rolling circle amplification (e.g., a φ29, a Bst, or a Vent exo-DNA polymerase), and (c) a recombinase. In some instances, the kit can further comprise one or more of: (a) an exonuclease; (b) a DNA polymerase; (c) a DNA ligase; (d) a restriction enzyme; and (e) a gyrase.

In some embodiments, the kits for producing a replicated linear RNA construct in a cell-free system comprise one or more of: (a) a template circular nucleic acid construct that comprises one or more of: (i) a target sequence, (ii) a promoter (e.g., a T7 promotor) adjacent to the 5′ end of the target sequence, and (iii) a Poly-A tail adjacent to the 3′ end of the target sequence, where the Poly-A tail comprises one or more restriction enzyme sites; (b) a polymerase configured to perform rolling circle amplification (e.g., a φ29, a Bst, or a Vent exo-DNA polymerase); (c) a restriction enzyme configured to cleave the template circular nucleic acid construct at the one or more restriction enzyme sites; and (d) an RNA polymerase configured to perform in vitro transcription (e.g., a T7 RNA polymerase).

In some embodiments, each enzyme used in the methods described herein is stored separately prior to use in the kit. In some embodiments, one or more enzymes used in the methods described herein are stored together prior to use in the kit.

In some embodiments, the kit can further comprise a suitable excipient, carrier, diluent, or any combination thereof. In some embodiments, the excipient, carrier, diluent, or any combination thereof can be any excipient, carrier, diluent, or any combination thereof known for storage of one or more of the enzymes disclosed herein.

### EXAMPLES

The following examples are included for illustrative purposes only and are not intended to limit the scope of the disclosure. It will be understood by those of skill in the art that numerous and various modifications can be made to yield essentially similar results without departing from the spirit of the present disclosure.

### Example 1: Amplification of Template Circular Nucleic Acid Constructs by Rolling Circle Amplification (RCA) for Production of Replicated Circular DNA

An exemplary plasmid (FIG. 1) is provided comprising a target sequence (bounded by Lox66 and Lox71 sites), a vector backbone (containing origin of replication and an antibiotic resistance gene to enable propagation in bacteria during the cloning process), and a unique restriction site in the vector backbone using conventional bacteria transformation and clonal expansion techniques to produce nanogram amounts of plasmid. An expression vector is modified to comprise the target sequence and the restriction enzyme sites. The plasmid is then used for in vitro cell-free replication. RCA is performed on a small scale (step a. in FIG. 2) using an amplification kit comprising a sample buffer to dilute template circular nucleic acid constructs and random hexamer primers to prime nucleic acid replication, a reaction buffer including salts, pH balance agents, and deoxynucleotides to support in vitro nucleic acid synthesis, and an enzyme mix including a cryoprotected mix of φ29 enzyme in glycerol. The template circular nucleic acid construct is amplified by mixing the following components in a PCR tube: 1 μl of the template DNA to be amplified, 5 μl of sample buffer, 5 μl of reaction buffer, and 0.2 μl of enzyme mix.

The sample is mixed by pipetting and incubated for 18 hours at 30° C. in a thermocycler. Following the incubation, the DNA polymerase is inactivated by heating at 65° C. for 10 minutes. The tube contains about a microgram of replicated nucleic acid constructs in concatenated form. The amplification reaction is scaled up in a suitable reaction vessel to produce larger quantities of plasmid on the gram or kilogram scale.

### Example 2: Restriction Digestion of Replicated Nucleic Acid Constructs

RCA-amplified nucleic acid constructs in concatenated form is digested with a restriction enzyme that cleaves uniquely within in the vector backbone (step b. of FIG. 2). This step serves two major purposes: 1) digesting the long, concatenated DNA reduces the viscosity of the solution (making further manipulations easier), and 2) it cleaves the vector backbone, which prevents recircularization of the vector backbone during a subsequent recombination step (see Example 3).

The heat-inactivated RCA reaction is subjected to restriction digestion by combining the following in a PCR tube: 11.2 μL RCA reaction (prepared in Example 1), 23.8 μL ddH2O, 4 μL 10× Buffer, and 1 μL restriction enzyme. The reaction is incubated at the appropriate temperature for the enzyme (typically 37° C.) for 5 hours.

Following digestion, the DNA can be purified via a number of different approaches to prepare it for the next step in the process, including: phenol/chloroform extraction with alcohol precipitation, agarose gel electrophoresis followed by gel extraction, agarose gel electrophoresis followed by electroelution, and/or no purification, other than heat treatment to inactivate the restriction enzyme.

Next, agarose gel electrophoresis followed by gel extraction is used to provide high-purity nucleic acid constructs for optimization of subsequent stages. The entire restriction digestion reaction is mixed with 8 μL of 6× of a gel loading dye, and loaded into a well of a 1% agarose gel prepared with TAE buffer. An adjacent well is loaded with a suitable nucleic acid ladder to enable size measurement of the nucleic acids in the sample well. The gel is run at 100 V for 1-2 hours, and then the band corresponding to the linear plasmid is excised from the gel with a razor blade and purified with a gel extraction kit following the manufacturer-provided protocol.

### Example 3: Re-Circularization of the Target Sequence Via Site-Specific Recombination

Cre recombinase is used to perform a recombination reaction that joins the Lox66 and Lox71 sites, circularizing the target sequence and cutting out the vector backbone (step c. of FIG. 2). The linearized purified nucleic acid from Example 2 is subjected to Cre-mediated recombination as follows: 1,000 ng of DNA, 5 μL 10× Cre Recombinase reaction buffer, ddH2O to 49 μL, and 1 μL Cre Recombinase 15,000 units/mL.

The reaction is incubated at 37° C. for 30 minutes and then 70° C. for 10 minutes. The reaction is analyzed by running on a 1% agarose gel made from TAE buffer.

### Example 4: Exonuclease Digestion of Non-Circular Nucleic Acids

Non-circular nucleic acids (corresponding to remnants of the vector backbone, or residual target nucleic acids that was not successfully circularized) is removed through exonuclease digestion (step d. of FIG. 2). An exonuclease mix designed to specifically target and degrade linear single- or double-stranded nucleic acids, while ignoring nicked or closed-circular nucleic acids, is used to remove the vector backbone or unsuccessfully circularized nucleic acids.

The nucleic acids isolated from Example 3 is subjected to the following conditions: 500 ng DNA (from Example 3), X μL sterile water, 2 μL 25 mM ATP, 5 μL 10× Reaction Buffer, 1 μL Plasmid-Safe DNase (10 U), and TOTAL VOLUME: 50 μL.

The reaction is incubated at 37° C. for 1 hour, followed by 70° C. for 30 minutes. The reaction is analyzed by running on a 1% agarose gel made from TAE buffer.

### Example 5: Polishing Step to Repair any Circularized Nucleic Acid Nicks

The circular nucleic acid constructs from the previous example is treated with an enzyme cocktail designed to repair any lesions in the DNA, such as nicks, gaps, or certain types of DNA damage (step e. of FIG. 2).

These repair functions are provided by a repair mix of Taq DNA Ligase, Endonuclease IV, Bst DNA Polymerase, Fpg, Uracil-DNA Glycosylase (UDG), T4 PDG (T4 Endonuclease V) and Endonuclease VIII.

A reaction is set up as follows: 1× buffer, 100 μM dNTPs, 1×NAD+, 50-500 ng of the circular nucleic acid constructs, X μL laboratory water, 1 μl of repair mix, and TOTAL VOLUME: 50 μL. The repair reaction was then incubated for 15-20 minutes at 37° C.

### Example 6: Polishing Step to Ensure Supercoiling

The circular product is treated with enzymes to repair any nicks in the DNA (DNA polymerase and DNA ligase), along with enzymes to catalyze supercoiling of the circular product (the enzymes gyrA/B) (step e. of FIG. 2).

DNA gyrase (a holoenzyme composed of gyrA and gyrB) is used to introduce supercoils into the final DNA product. The supercoiling is thought to help with improving the performance of the plasmid DNA in downstream applications such as transfection.

E. coli DNA Gyrase Enzyme is used to supercoil the resulting material as follows: 200 ng of DNA from Example 5, 6 μL of 5× assay buffer, X μL of laboratory water, 1 U of DNA Gyrase Enzyme, and TOTAL VOLUME: 30 μL.

Following the reaction, the sample is run on an agarose gel in the absence of ethidium bromide. The gel is stained after running and visualized to inspect the degree of supercoiling.

### Example 7: Production of a Template Circular Nucleic Acid Construct from a Linear DNA Template

This example describes production of a template circular nucleic acid construct from a linear DNA template, which can be utilized for production of a replicated linear RNA construct. An exemplary linear DNA template is provided comprising, from 5′ to 3′, a T7 promotor, a target sequence (e.g., a gene of interest), a Poly-A tail, and a unique restriction site that cleaves at the 3′ end of the Poly-A tail (FIG. 3).

A linear DNA template corresponding to FIG. 3 is chemically synthesized with type II restriction sites provided for production of compatible overhangs at the 5′ and 3′ ends of the linear DNA template (e.g., BsaI). The linear DNA template is then digested with BsaI and the linear DNA template is ligated to produce a circular DNA template by Golden Gate assembly. While a single linear DNA template is utilized in this example, individual chemically synthesized linear DNA templates can be combined modularly and circularized using Golden Gate assembly by including a unique type II restriction enzyme site on each end of two synthesized linear DNA constructs to be joined, which can be carried out in a modular fashion with assembly of each linear DNA construct.

Production of the replicated circular DNA construct described in Examples 1-6 above also utilize a template circular nucleic acid construct. While the particular template circular nucleic acid construct provided in this example is utilized for production of replicated linear RNA due to the arrangement of the replication cassette in the template, the method provided in this example can be utilized to produce a template circular nucleic acid construct from a linear DNA template that can be used for producing a replicated circular DNA construct by modifying the linear DNA template to include the recombination sites, restriction sites, and backbone sequence arrangement described in Example 1 and depicted in FIG. 1.

### Example 8: Amplification of Template Circular Nucleic Acid Constructs by Rolling Circle Amplification (RCA) for Production of Replicated Linear RNA

The exemplary linear DNA template depicted in FIG. 3 is utilized to produce a template circular DNA construct for rolling circle amplification using the same protocol described above in Example 1. As shown in FIG. 5, RCA of the template circular DNA produced in Example 7 (depicted as “Input DNA” in FIG. 5) produces a circular concatenated DNA replication product with the restriction enzyme site present adjacent to the Poly-A tail included in each concatemeric repeat (a linear representation of the circular concatenated DNA replication product is shown in FIG. 5 for simplicity).

### Example 9: Restriction Enzyme Digestion of Replication Product

The RCA-amplified nucleic acid construct in concatenated form produced in Example 8 is digested with a restriction enzyme that site-specifically and uniquely cleaves at the restriction site present adjacent to the Poly A tail of each concatemeric repeat. This step results in cleavage of a single copy of the concatenated replication product into multiple copies of the linear DNA template, thus resulting in replicated template that can be utilized for in vitro transcription.

Following digestion, the linear DNA can be purified as described above in Example 2. Where the final desired product is replicated linear DNA, the linear DNA construct purified as described above constitutes the finalized desired replicated linear DNA product. Where the final desired product is replicated linear RNA or circular RNA, the linear RNA can be produced according to Example 10 below, and circular RNA can be produced according to Example 11 below.

### Example 10: Production of Replicated Linear RNA Via In Vitro Transcription (IVT)

The replicated linearized DNA template produced in Example 9 is used for production of linear RNA constructs via IVT.

A reaction is set up as follows: 100 mM dNTPs, 2 μL standardized T7 RNA polymerase mix containing T7 RNA polymerase, 1 μg of the replicated linearized DNA template produced in Example 9, and nuclease-free water. The reaction mixture is mixed thoroughly and incubated for 2 hours at 37° C.

Following IVT, the reaction mixture is diluted in nuclease-free water and 2 μL of DNase I (RNase-free) is added to remove residual template DNA. The reaction mixture is incubated for 15 minutes at 37° C. The linear RNA construct is purified using an RNA cleanup kit and analyzed by gel electrophoresis to assess purity.

### Example 11: Production of Replicated Circular RNA Via In Vitro Circularization

The replicated linearized RNA product produced in Example 10 is used for production of circular RNA constructs.

A reaction is set up as follows: 10-50 mM ATPs, 1 uL T4 RNA ligase, 10% PEG8000, 1 μg of the replicated linear RNA product produced in Example 10, and reaction buffer (50 mM Tris-HCl, pH 7.5, 10 mM MgCl2, 1 mM DTT). The reaction mixture is mixed thoroughly and incubated for 2 hours at 25° C.

The reaction mixture is boiled to terminate the reaction, and then diluted in nuclease-free water. Poly(A) polymerase is added followed by RNase R digestion in the presence of a Li+ containing buffer to remove residual linear RNA product. The circular RNA construct is purified using an RNA cleanup kit and analyzed by gel electrophoresis to assess purity.

### Example 12: Rolling Circle Amplification (RCA) of Plasmid DNA

In this example, a circular plasmid DNA template was amplified many fold via rolling circle amplification to generate concatenated copies of the plasmid. The template plasmid was subjected to rolling circle amplification (RCA) under the following conditions using the Cytiva TempliPhi Large Construct kit.

A reaction was set up as follows: 12.25 uL Sample Buffer, 13.75 uL Reaction Buffer, 2.75 uL dNTP solution (NEB, N0447L), 1 uL plasmid template (10 ng/uL), 1 uL RCA enzyme. The reaction was mixed thoroughly, and incubated for 16 hours at 30° C., followed by heat inactivation at 65° C. for 10 min, followed by hold at 12° C.

### Example 13: Linearization of RCA-Amplified Plasmid Concatenate

The concatenated material produced in Example 11 was processed into linear, monomeric plasmid following a restriction digestion process. In this example, the concatenated product of a plasmid amplified via RCA was treated with a restriction enzyme to digest the concatenated product into linear copies of the template plasmid DNA.

The product of the RCA reaction was treated under the following conditions: 30.75 uL RCA (entire RCA reaction), 57.25 uL ddH2O, 10 uL 10× Cutsmart buffer (NEB), 2 uL EcoRI-HF (NEB). The reaction incubated for 2 hours at 37° C. After two hours 1 μL of additional EcoRI enzyme was added and the reaction was continued for an additional hour.

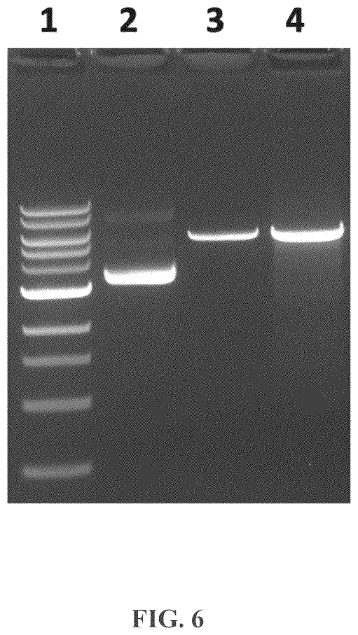

Following a successful RCA reaction, this linearization process generated material that was indistinguishable from the linearized form of the starting template plasmid as seen by comparing lanes 3 and 4 in the gel in FIG. 6. FIG. 6 lane depicts the 1 kb ladder (NEB), lane 2 depicts supercoiled template plasmid, lane 3 depicts the linearized template plasmid, lane 4 depicts the RCA-amplified and linearized template plasmid. As can be seen by comparing the size of DNA in lanes 3 and 4, the RCA-amplified and linearized material has the same molecular weight as the linearized template material.

### Example 14: Recombination of RCA-Amplified and Linearized Material to Generate Circular Plasmid

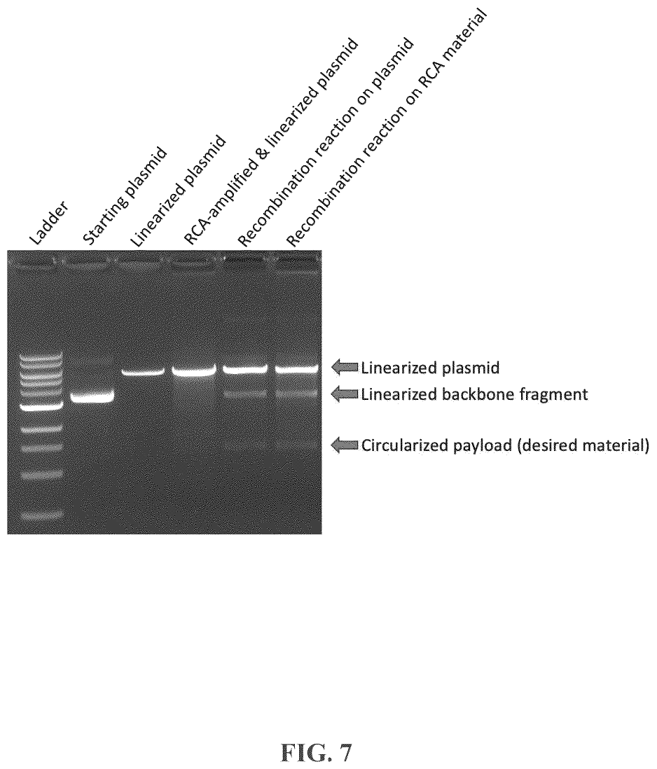

In this example, RCA-amplified and linearized material was treated with Cre recombinase to re-circularize the plasmid while simultaneously removing the unnecessary plasmid backbone elements. Lox66 and lox71 sites were strategically placed to flank the plasmid backbone. Upon recombination with Cre, the lox66 and lox71 sites recombined to generate a circular product containing the plasmid payload, and a linear product containing the plasmid backbone (FIG. 7).

The CRE recombination reaction was performed as follows: 1.5 uL 10× Cre buffer (NEB), 11.5 uL ddH2O, 263 ng RCA amplified and linearized plasmid, 1 uL Cre enzyme (NEB). The reaction was run at 37° C. for 30 min, followed by inactivation at 70° C. for 10 min. The products from the reaction was run on an agarose gel to visualize the products (FIG. 7). FIG. 7 lane 1 depicts the 1 kb ladder (NEB), lane 2: depicts the supercoiled template plasmid, lane 3 depicts the linearized template plasmid, lane 4 depicts the RCA-amplified and linearized template plasmid, lane 5 depicts the Cre recombination reaction performed on linearized plasmid template, and lane 6 depicts the Cre recombination reaction performed on RCA-amplified and linearized plasmid.

### Example 15 Repair and Supercoiling of Nicked Plasmid DNA

In this example, supercoiled plasmid DNA that was damaged via nicking of the DNA was repaired and subjected to enzymatic supercoiling.

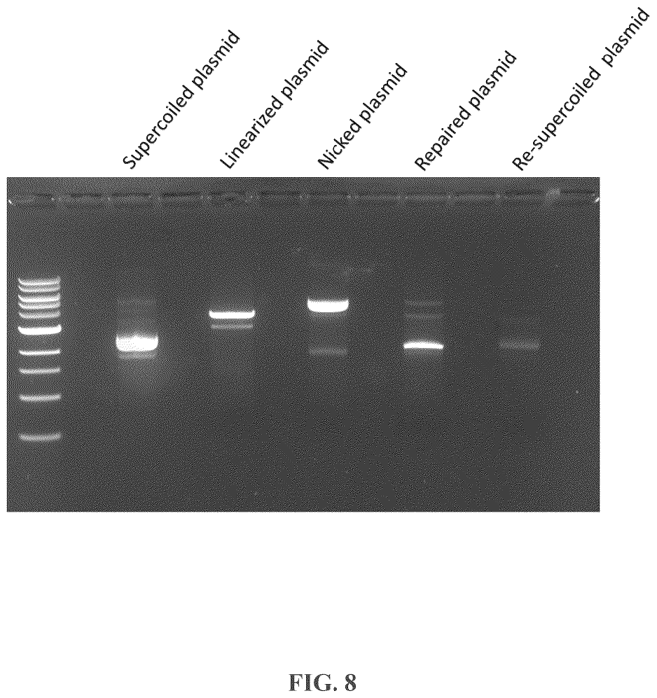

To simulate damaged DNA due to nicks, supercoiled plasmid DNA was treated with a nickase enzyme to generate nicked plasmid DNA material for use in optimizing repair reactions as follows: 5 uL Plasmid DNA (1 ug total), 5 uL 10× r3.1 buffer (NEB), 39 uL ddH2O, 1 uL Nt.BspQI Nickase (NEB). The reaction was at 50° C. for 1 hour followed by 80° C. incubation for 20 min to heat inactivate enzyme. When run on an agarose gel, the nicked plasmid ran higher than supercoiled or linearized forms of plasmid DNA (FIG. 8).

To repair the nicked DNA, the DNA was subjected to the following nick repair reaction: 20 uL nicked plasmid DNA, 5 uL 10× Thermopol Buffer (NEB), 0.5 uL NAD+(NEB), 0.5 uL dNTP (NEB), 23 uL ddH2O, 1 uL PreCR Mix (NEB). The reaction was incubated at 37° C. for 20 min. When run on an agarose gel, the previously nicked plasmid was comparable to intact, circular plasmid DNA as shown on agarose gel (FIG. 8).

The plasmid DNA was expected to have lost its supercoiling upon being nicked. To introduce supercoils back into the plasmid DNA, the material was subjected to the following supercoiling reaction: 20 uL post-PreCR reaction, 10 uL Assay Buffer (TopoGEN), 19 uL ddH2O, 1 uL Gyrase (TopoGEN). The reaction was incubated for 37° C. for 1 hour. Material was run on an agarose gel to confirm that its migration pattern matches supercoiled plasmid DNA (FIG. 8). FIG. 8 lane 1 depicts the 1 kb ladder (NEB), lane 2 depicts the supercoiled template plasmid, lane 3 depicts the linearized template plasmid, lane 4 depicts the nicked plasmid DNA, lane 5 depicts repaired plasmid DNA post PreCR repair reaction, and lane 6 depicts the material that has been subjected to supercoiling reaction.

While preferred embodiments of the present disclosure have been shown and described herein, such embodiments are provided by way of example only. Numerous variations, changes, and substitutions will occur without departing from the present disclosure. It should be understood that various alternatives to the embodiments described herein may be employed. It is intended that the following claims define the scope of the present disclosure and that methods and structures within the scope of these claims and their equivalents be covered thereby. The above examples were obtained by using the following materials and methods.

1. An in vitro method of producing a replicated circular DNA construct or a replicated linear RNA construct from a template circular nucleic acid construct in a cell-free system, the method comprising:
(a) providing the template circular nucleic acid construct that comprises:
(i) a target sequence comprising one or more ends;
(ii) one or more enzyme recognition sites flanking the one or more ends of the target sequence; and
(iii) a backbone sequence;
(a) contacting the template circular nucleic acid construct with a DNA polymerase configured to perform rolling circle amplification of the template circular nucleic acid via the DNA polymerase, thereby forming a replication product that comprises concatemeric replications of the target sequence and the backbone sequence joined together; and
(b) contacting the replication product with an enzyme, thereby producing the replicated circular DNA construct or the replicated linear RNA construct from the replication product; wherein:
(i) when the one or more enzyme recognition sites comprise recombination sites that flank each end of the target sequence and the enzyme is a recombinase that comprise a LoxP sequence, a Lox66 sequence, a Lox71 sequence, a FRT sequence, or a functional variant thereof, the contacting results in recombination of the replication product, which removes the backbone sequence from the replication product and joins the ends of the target sequence, thereby producing the replicated circular DNA construct; and
(ii) when the one or more enzyme recognition sites comprise one or more restriction sites flanking one or more ends of the target sequence and the enzyme is a restriction enzyme that cleaves the replication product at the one or more restriction sites, the contacting results in formation of a restriction enzyme-digested replication product, and the method further comprises contacting the restriction enzyme-digested replication product with an RNA polymerase configured to perform in vitro transcription, thereby producing the replicated linear RNA construct.

2. The method of claim 1, wherein the DNA polymerase is a φ29, a Bst, or a Vent exo-DNA polymerase.

3.-9. (canceled)

10. The method of claim 1, wherein the recombinase is a Cre recombinase, an FLP recombinase, a lambda phage integrase, a TP901-1 lactococcal phage recombinase, a Bxb1 integrase, an R4 integrase, a Xer recombinase, a Dre recombinase, or a φC31 integrase.

11.-12. (canceled)

13. The method of claim 1, further comprising contacting the replication product with a restriction enzyme that selectively cleaves the backbone sequence prior to contacting the replication product with the recombinase, thereby forming a digested replication product for contacting with the recombinase.

14. The method of claim 1, further comprising contacting the replication product with an exonuclease after contacting the replication product with the recombinase, wherein the exonuclease selectively digests the cleaved backbone sequence.

15. The method of claim 1, further comprising contacting the replicated circular DNA construct with a composition that comprises one or more of:
a. a second DNA polymerase,
b. a DNA ligase,
c. a restriction enzyme, or
d. a gyrase,
wherein the contacting is in an amount and duration sufficient to repair nicks in the replicated circular DNA construct and introduce supercoiling in the replicated circular DNA construct.

16. The method of claim 1, further comprising:
a. contacting the replicated circular DNA construct with a composition that comprises a second DNA polymerase, and a DNA ligase, wherein the contacting is in an amount and duration sufficient to repair nicks in the replicated circular DNA construct; and
b. contacting the replicated circular DNA construct with a composition that comprises a gyrase, wherein the contacting is in an amount and duration sufficient to introduce supercoiling in the replicated circular DNA construct.

17. The method of claim 1, wherein the one or more enzyme recognition sites comprise one or more restriction sites that flank one or more ends of the target sequence, and wherein the enzyme is the restriction enzyme, thereby producing the replicated linear RNA construct.

18. The method of claim 17, wherein the template circular nucleic acid sequence further comprises a Poly-A tail sequence adjacent to the 3′ end of the target sequence.

19. The method of claim 18, wherein the one or more restriction sites are present on the Poly-A tail sequence.

20. The method of claim 17, wherein the template circular nucleic acid sequence further comprises a promoter configured to interface with the RNA polymerase.

21. The method of claim 20, wherein the RNA polymerase is a T7 RNA polymerase, and wherein the promoter is a T7 promoter.

22.-25. (canceled)

26. A kit for replicating a circular DNA construct in a cell-free system, the kit comprising:
a. a template circular nucleic acid construct that comprises:
i. a target sequence flanked by recombination sites on each end, wherein the target sequence is configured to form the circular nucleic acid construct upon recombination, and
ii. a backbone sequence;
b. a φ29, a Bst, or a Vent exo-DNA polymerase; and
c. a recombinase.

27. The kit of claim 26, further comprising one or more of:
a. an exonuclease;
b. a DNA polymerase;
c. a DNA ligase;
d. a restriction enzyme; and
e. a gyrase.

28. The kit of claim 26, wherein the recombinase is a Cre recombinase, an FLP recombinase, a lambda phage integrase, a TP901-1 lactococcal phage recombinase, a Bxb1 integrase, an R4 integrase, a Xer recombinase, a Dre recombinase, or a φC31 integrase.

29. A kit for replicating a linear RNA construct in a cell-free system, the kit comprising:
a. a template circular nucleic acid construct that comprises:
i. a target sequence,
ii. a T7 promoter adjacent to the 5′ end of the target sequence, and
iii. a Poly-A tail adjacent to the 3′ end of the target sequence, wherein the Poly-A tail comprises one or more restriction enzyme sites;
b. a φ29, a Bst, or a Vent exo-DNA polymerase;
c. a restriction enzyme configured to cleave the template circular nucleic acid construct at the one or more restriction enzyme sites; and
d. a T7 RNA polymerase.

30.-62. (canceled)

63. The method of claim 1, wherein the template circular nucleic acid construct is a single-stranded template circular DNA construct.

64. (canceled)

65. The method of claim 63, wherein the method further comprises producing the single-stranded template circular DNA construct from a template linear DNA construct prior to 1(b); and wherein the template linear DNA construct comprises a telRL sequence on each side of the target sequence, and wherein the single-stranded template circular DNA construct is produced from the template linear DNA construct prior to (a) by contacting the template linear DNA construct with a telN protelomerase.

66. The method of claim 1, wherein the template circular nucleic acid construct is a double-stranded template circular DNA construct.

67. (canceled)

68. The method of claim 66, wherein the method further comprises producing the double-stranded template circular DNA construct from a template linear DNA construct that comprises one or more ends prior to 1(b) by ligation of the ends of the template linear DNA construct.

69. (canceled)
